(19) United States
(12) Patent Application Publication
(10) Pub. No.: US 2004/0002069 A1
(43) Pub. Date: Jan. 1, 2004

(54) Hepatic gene expression profiling of streptozotocin-induced diabetes

(75) Inventors: Stephen R. Spindler, Riverside, CA (US); Joseph M. Dhahbi, Riverside, CA (US); Patricia L. Spindler, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/177,958

(22) Filed: Jun. 21, 2002

(51) Int. Cl. C12Q 1/68

(52) U.S. Cl. 435/6

(57) ABSTRACT

This invention is based on the discovery that certain genes have expression levels that are substantially increased or decreased in the liver of animals with diabetes. Monitoring the expression levels of such genes allows diagnosis of diabetes. In certain embodiments, the invention also provides methods of modulating the expression levels of such genes to treat diabetes and diabetic complications. In other embodiments, the invention provides a screening assay that identifies compounds which modulate the expression level of polynucleotides or functional properties of polypeptides that are over or under expressed in diabetes. In some cases, these compounds can be used to treat diabetes and diabetic complications. In still other embodiments, the invention provides assays to determine whether compounds intended for treatment of other diseases also induce diabetes.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Diabetes is a common metabolic disorder in humans where the body is unable to properly regulate glucose levels. Normally, when glucose levels rise in the blood of healthy individuals, the pancreas produces insulin, which causes the muscle and fat cells to extract sugar from the blood and the liver to stop producing glucose. However, in diabetes the body is unable to reduce glucose levels. There are two primary types of diabetes. Type I diabetes, commonly known as juvenile-onset diabetes, arises due to destruction of the insulin-producing islet cells of the pancreas. Type II diabetes, sometimes termed insulin resistance, arises from a defect in the signalling pathway of the insulin receptor; although there is sufficient insulin, binding of insulin to the receptors does not initiate the complex series of events that maintains glucose homeostasis.

Diabetes is associated with significant morbidity and mortality, and is a contributor to the development of other diseases. The major complications associated with diabetes are the development of increase susceptibility to xenobiotic-induced cytotoxicity, microvascular disorders of the retina, kidney and peripheral nerves (Wang et al., Toxicol. Appl. Pharmacol. 166:92-100 (2000); J. W. Baynes, Diabetes 40:405-412 (1991)). In addition, abnormalities of the digestive system are not uncommon during diabetes (see, Feldman & Schiller, Ann. Intern. Med 98:378-384 (1998). The development of diabetes is also accompanied by the development of hepatic biochemical and functional abnormalities. These include alterations in carbohydrate, lipid and protein metabolism, as well as changes in antioxidant status (Shmueli et al., Baillieres Clin. Endocrinol. Metab. 6:719-743 (1992); Chatila & West, Medicine 75:327-333(1996); Feingold, et al., Diabetes 31:388-395 (1982); McLennan, et al., Diabetes 40:344-348 (1991); Saxena, et al., Biochem. Pharmacol. 45:539-542 (1993)). These hepatic abnormalities are of particular importance because of their potential role in the systemic effects of liver function.

In view of the above, it is clear that there is a need for methods for detecting diabetes, methods for treating diabetes, screening assays for compounds that treat diabetes, pharmaceutical compositions identified by such methods, as well as assays to ensure that compounds for treatment of other diseases do not induce diabetes. In view of the potential for hepatic abnormalities induced by diabetes, it is also clear that there is particular need for the identification of genes that are over- or under-expressed in the liver of diabetic animals. This invention fulfills this and other needs.

### BRIEF SUMMARY OF THE INVENTION

The present invention provides EST and genes with either increased or decreased expression in the liver of mammals with diabetes.

In one aspect, the invention provides a method of detecting diabetes in a patient comprising detection of the expression level of at least one of the polynucleotides listed in Table 1. Typically, the method further comprises detection of the expression level of a second polynucleotide listed in Table 1. In certain embodiments, the method comprises detection of polynucleotides including, but not limited to, a histidine lysase gene, a beta CCAAT/enhancer binding protein (CC/EBP), solute carrier family 22 (member 1), Cyt P450 1a2 (aromatic compound), and a corticosteroid binding protein. Typically, the expression level is detected by an oligonucleotide array or a Northern blot. The detected diabetes can usually be classified as either Type I diabetes or Type II diabetes.

In another aspect, the invention provides a method of treating diabetes or diabetic complications which comprises modulating the expression level of at least one polynucleotide listed in Table 1. Typically, the diabetes is Type II diabetes. The treated diabetic complications can include, but are not limited to, alterations in xenobiotic metabolism, retinopathy, nephropathy, neuropathy, and hepatic functional abnormalities. Usually, the diabetic complications are hepatic functional abnormalities. These hepatic functional abnormalities can include, but are not limited to, loss of hepatic insulin sensitivity; excessive protein breakdown; reduction of protein synthesis; excessive hepatic glucose production (gluconeogenesis); reduction in cell growth and regeneration; excessive cell death (apoptosis); serum dyslipidemia resulting from altered hepatic lipid and protein metabolism; altered drug, metabolite, and xenobiotic metabolism; enhanced oxidative damage and cellular stress; and enhanced inflammatory and disregulated immune responsiveness. In certain embodiments of the methods, the modulation of expression levels for treating diabetes comprises reducing the expression level of the polynucleotide using anti-sense molecules, ribozymes, and small interfering RNAs. In other embodiments, the expression level of the polynucleotide is increased.

In another aspect, the invention provides a method of treating diabetes or diabetic complications, wherein the method comprises modulating the activity of at least one polypeptide encoded by a polynucleotide listed in Table 1.

In yet another aspect, this invention provides an assay for identifying a compound that modulates a polypeptide encoded by a polynucleotide listed in Table 1. Typically, the assay comprises the steps of: (a) contacting a compound with the polypeptide; and (b) determining the functional effect of the compound upon the polypeptide.

In still yet another aspect, the invention provides a high-throughput drug screening assay comprising the steps of: (a) contacting a cell isolated from a mammal with streptozotocin-induced diabetes with a test compound; (b) measuring the expression level of a polynucleotide listed in Table 1; and (c) comparing the expression level of the polynucleotide in a cell contacted with a test compound to the expression level of the polynucleotide in a cell from a control mammal, wherein the test compound that modulates the expression level of the polynucleotide is a candidate for the treatment of diabetes or diabetic complications.

In another aspect, the invention provides a drug screening assay comprising the steps of: (a) administering a test compound to a mammal with streptozotocin-induced diabetes; (b) measuring the expression level of a polynucleotide encoding a polypeptide selected from the group consisting of: polypeptides listed in Table 1; and (c) comparing the expression level of the polynucleotide in a mammal contacted with a test compound to the expression level of the polynucleotide in a control mammal; wherein the test compound that modulates the expression level of the polynucleotide is a candidate for the treatment of diabetes or diabetic complications.

In another aspect, the invention provides a high-throughput assay for determining whether a test compound induces diabetes comprising the steps of: (a) contacting a cell isolated from a mammal with a test compound; (b) measuring the expression level of a polynucleotide listed in Table 1; and (c) determining whether the test compound modulates the expression level of the polynucleotide in the same manner as diabetes, wherein the test compound that modulates the expression level of the polynucleotide in the same manner as diabetes is a compound that induces diabetes.

In another aspect, the invention provides an assay for determining whether a test compound induces diabetes, comprising the steps of: (a) administering a test compound to a mammal; (b) measuring the expression level of a polynucleotide listed in Table 1; and (c) determining whether the test compound modulates the expression level of the polynucleotide in the same manner as diabetes, wherein the test compound that modulates the expression level of the polynucleotide in the same manner as diabetes is a compound that induces diabetes.

In still yet another aspect, this invention provides a pharmaceutical composition comprising a compound identified by the above described assays together with a physiologically acceptable excipient.

### Definitions

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single letter codes.

The term “expression level” as used herein encompasses both the “gene expression level” and the “protein expression level” of a particular polynucleotide sequence. “Gene expression level” refers to amount of mRNA which is transcribed from a polynucleotide. Typically, gene expression levels are measured by quantitating mRNA levels via oligonucleotide assays, Northern blots, PCR-based amplification assays, etc. “Protein expression level” refers to the amount of protein which is translated from the mRNA which is transcribed from a polynucleotide.

The term “streptozotocin-induced diabetes” as used herein refers to diabetes that has been artificially induced with streptozotocin (a.k.a. streptozocin), a methyl nitroso urea with a 2 substituted glucose (the compound is produced by Streptomyces achromogenes). Streptozotocin has toxic effects on both the insulin-producing β-cells of the pancreas and insulin-dependent signalling, thus administration of the compound to an animal induces a model with characteristics of both Type I and Type II diabetes.

The term “induces diabetes” as used herein does not require that the particular process or event induces all aspects of diabetes. Instead, it refers to any process or event that induces certain effects of diabetes or mimics certain effects of diabetes.

The term “hepatic functional abnormalities” as used herein refers to any variation from normal liver metabolism. These variations can include, but are not limited to, alterations in carbohydrate metabolism, lipid metabolism, protein metabolism, glutathione metabolism, antioxidant status, etc. Typically, these variations are due to changes in the levels or activity of liver enzymes.

As described herein, the term “diabetes-associated protein”, “diabetes protein”, “diabetes-associated sequence”, “diabetes sequence”, and “diabetes-specific nucleic acid” refer to nucleotide and/or protein sequences that are either overexpressed or underexpressed in animals with diabetes or diabetes-like conditions compared to-animals without diabetes. The terms also encompass nucleic acid and polypeptide polymorphic variants, alleles, mutants, and interspecies homologs that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of at least about 25, 50, 100, 200, 500, 1000, or more nucleotides, to a nucleotide sequence of or associated with a polynucleotide disclosed in Table 1; (2) bind to antibodies, e.g., polyclonal antibodies, raised against an immunogen comprising an amino acid sequence encoded by a nucleotide sequence of or associated with a polynucleotide disclosed in Table 1, and conservatively modified variants thereof; (3) specifically hybridize under stringent hybridization conditions to a nucleic acid sequence, or the complement thereof of Table 1 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a polynucleotide sequence of Table 1. A polynucleotide or polypeptide sequence is typically from a mammal including, but not limited to, primate, e.g., human; rodent, e.g., rat, mouse, hamster; cow, pig, horse, sheep, or other mammal. The polypeptides and polynucleotide can either be naturally occurring or recombinant.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then the to be “substantially identical.” This definition also refers to, or may be applied to, the complement of a test sequence. The definition also includes sequences that have deletions and/or additions, as well as those that have substitutions, as well as naturally occurring, e.g., polymorphic or allelic variants, and-man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a-“conservatively modified variant” where the alteration results-in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid-backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. As used herein, the term “polynucleotide” refers to genes, cDNAs, expressed sequences tags (ESTs), etc.

Peptide nucleic acids (PNA) include peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each-containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes can directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to essentially no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications (1990).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “functional effects” in the context of assays for testing compounds that modulate activity of a diabetes protein includes the determination of a parameter that is indirectly or directly under the influence of the diabetes protein or nucleic acid, e.g., an enzymatic, functional, physical, or chemical effect, such as the ability to reduce the severity of diabetes. It includes an ability to properly metabolize glucose. “Functional effects” include in vitro, in vivo, and ex vivo activities.

By “determining the functional effect” is meant assaying for a compound that increases or decreases a parameter that is indirectly or directly under the influence of a diabetes protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the diabetes protein; measuring binding activity or binding assays, e.g., binding to antibodies or other ligands. Determination of the functional effect of a compound on diabetes in a animal can also be performed using clinical tests known to those of skill in the art such as the fasting plasma glucose test, nonfasting plasma glucose test, oral glucose tolerance test, etc. Functional effects can also be determined by measuring the level of insulin secretion by β-cells in the pancreas or the responsiveness of the insulin receptor signaling pathway. These can be done via any method known to those of skill in the art. For example, functional effects on insulin receptor signaling can be evaluated by detection of signaling intermediates, measurement of changes in RNA or protein levels of proteins in the pathway, detection of reporter gene expression (CAT, luciferase, β-gal, GFP and the like) in in vitro mammalian cell cultures expressing insulin receptor, e.g., via chemiluminescence, fluorescence, colorimetric reactions, antibody binding, inducible markers, and ligand binding assays.

“Inhibitors”, “activators”, and “modulators” of diabetes polynucleotide and polypeptide sequences are used to refer to activating, inhibitory, or modulating molecules or compounds identified using in vitro and in vivo assays of diabetes polynucleotide and polypeptide sequences of the invention. Inhibitors are compounds that, e.g., bind to, partially or totally block activity, decrease, prevent, delay activation, inactivate, desensitize, or down regulate the activity or expression of diabetes proteins of the invention, e.g., antagonists. Antisense nucleic acids may seem to inhibit expression and subsequent function of the protein. “Activators” are compounds that increase, open, activate, facilitate, enhance activation, sensitize, agonize, or up regulate diabetes activity. Inhibitors, activators, or modulators also include genetically modified versions of diabetes proteins, e.g., versions with altered activity, as well as naturally occurring and synthetic ligands, antagonists, agonists, antibodies, small chemical molecules and the like. Such assays for inhibitors and activators include, e.g., expressing the diabetes protein in vitro, in cells, or cell membranes, applying putative modulator compounds, and then determining the functional effects on activity, as described above. Activators and inhibitors of diabetes can also be identified by incubating diabetes cells with the test compound and determining increases or decreases in the expression of 1 or more diabetes proteins, e.g., 1, 2, 3, 4, 5, 10, 15, 20, 25, 30, 40, 50 or more diabetes proteins, such as diabetes proteins encoded by the sequences set out in Table 1.

Samples or assays comprising diabetes proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of a diabetes polypeptide is achieved when the activity value relative to the control (untreated with activators) is 110%, more preferably 150%, more preferably 200-500% (i.e., two to five fold higher relative to the control), more preferably 1000-3000% higher.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab′)₂, a dimer of Fab which itself is a light chain joined to VH-CHI by a disulfide bond. The F(ab′)₂ may be reduced under mild conditions to break the disulfide linkage in the hinge region, thereby converting the F(ab′)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which, e.g, (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

### BRIEF DESCRIPTION OF THE DRAWINGS

FIG. 1 illustrates the Northern blot validation of the array data. The expression of 10 genes was analyzed using Northern blots. Shown are the results for 5 genes which changed between streptozotocin (STZ) and control mice using Affymetrix microarrays. For Northerns, RNA samples from 6 mice, including the 3 used for the microarray studies, were analyzed from each experimental group. The hatched bars, representing the Northern data, are average fold changes of pairwise comparisons for the STZ and control mice. The solid bars, representing the microarray data, are the average fold changes in the specific mRNA derived from all possible pairwise comparisons among individual mice from STZ and control groups. The genes are identified by their respective GenBank numbers.

### DETAILED DESCRIPTION OF THE INVENTION

I. Introduction

This invention is based on the discovery that certain genes have expression levels that are substantially increased or decreased in the liver of animals with diabetes. In certain embodiments, the genes are ones associated with cytoprotective stress responses; oxidative and reductive xenobiotic metabolism; growth and signal transduction; carbohydrate, fat, and protein metabolism and homeostasis; and antioxidant defense. In some embodiments, the genes are those listed in Table 1. In particular, the genes can encode a histidine lysase gene, a beta CCAAT/enhancer binding protein (CC/EBP), a solute carrier family 22 (member 1), a Cyt P450 1a2 (aromatic compound), or a corticosteroid binding protein.

TABLE 1
Effects of SID on hepatic gene expression
The numbers in the FC column represent the average fold of the
streptozotocin-induced diabetes (SID)-related increase or
decrease in specific mRNA derived from all nine possible
pairwise comparisons among individual mice from STZ and
control groups (n = 3).

| | | | |
|---|---|---|---|
| GenBank | FC | Abbreviation | Gene/EST name |

Metabolism

| | | | |
|---|---|---|---|
| AA237297 | 2.0 | As1 | Argininosuccinate lyase |
| W08392 | 1.9 | Atp5f1 | ATP synthase, H+ transporting, |
| | | | mitochondrial F0 |
| | | | complex, subunit b, isoform 1 |
| AA096813 | 2.5 | Ctsl | CathepsinL |
| AA267683 | −2.0 | NF | Isopentenyl-diphosphate |
| | | | delta-isomerase |
| AA277720 | 2.5 | NF | NADH dehydrogenase |
| | | | (ubiquinone) 1 beta |
| | | | subcomplex 6 |
| W13878 | 1.9 | NF | DEAD/H (Asp-Glu-Ala- |
| | | | Asp/His) box polypeptide 1 |
| AA048927 | 3.6 | NF | Bifunctional aminoacyl- |
| | | | tRNA synthetase |
| AA106783 | 2.0 | Pabpc1 | Poly A binding protein, |
| | | | cytoplasmic 1 |
| AA110781 | 3.6 | Pck1 | Phosphoenolpyruvate |
| | | | carboxykinase 1, cytosolic |
| AA008321 | 2.0 | Psma4 | Proteasome (prosome, |
| | | | macropain) subunit, alpha |
| | | | type 4 |
| AA165782 | 1.9 | Psme1 | Protease (prosome, |
| | | | macropain) 28 subunit, alpha |
| AA289858 | −4.6 | Rnase4 | Ribonuclease, Rnase A family 4 |
| AA137436 | −14.0 | Sed1 | Stearoyl-Coenzyme A |
| | | | desaturase 1 |
| W53390 | 2.0 | Sdha | Succinate dehydrogenase |
| | | | complex, subunit A, |
| | | | flavoprotein (Fp) |
| AA277153 | 1.8 | Sf3b1 | Splicing factor 3b, |
| | | | subunit 1, 155 kDa |
| AA388848 | 1.9 | Sfrs5 | Splicing factor, arginine/ |
| | | | serine-rich 5 (SRp40, |
| | | | HRS) |

Growth Factors and Signal Transducers

| | | | |
|---|---|---|---|
| W98255 | −2.9 | Cd81 | CD 81 antigen |
| AA271265 | 2.0 | Creg | Cellular repressor of |
| | | | E1A-stimulated genes |
| AA028770 | 2.0 | Csrp2 | Cysteine-rich protein 2 |
| AA174883 | 1.8 | Edr | Erythroid differentiation |
| | | | regulator |
| W12681 | −2.8 | Hgfac | Hepatocyte growth factor |
| | | | activator HGFA |
| AA608036 | 1.9 | Ifrg15-pending | Interferon alpha responsive |
| | | | gene, 15 kDa |
| AA518955 | 1.9 | Rab9 | RAB9, member RAS |
| | | | oncogene family |
| AA472476 | −2.0 | Sirt2 | Sirtuin 2 (silent mating |
| | | | type information |
| | | | regulation 2, homolog) |
| | | | 2 (S. cerevisiae) |

Stress Response and Xenobiotic Metabolism

| W91222 | 2.4 | Cox7a3 | Cytochrome c oxidase, |
|---|---|---|---|
| | | | subunit VIIa 3 |
| AA137659 | −2.5 | Cyp2c40 | Cytochrome P450, 2c40 |
| AA138378 | 3.5 | Hspa9A | Heat shock protein, 74 kDa, A |
| W18904 | 1.8 | Tbca | Tubulin cofactor a |
| | | Nuclear and transcription factors | |
| AA217659 | 1.8 | Ncoa4 | Nuclear receptor coactivator 4 |
| AA270796 | 2.1 | Atf5 | Activating transcription factor 5 |
| AA390043 | 2.1 | Ncoa3 | Nuclear receptor coactivator 3 |
| AA600461 | 2.1 | Rnf4 | Ring finger protein 4 |
| AA016424 | 2.1 | Xbp1 | X-box binding protein 1 |

Miscellaneous

| | | | |
|---|---|---|---|
| AA172909 | 1.8 | Ccr4 | Carbon catabolite repression |
| | | | 4 homolog (S. cerevisiae) |
| W13835 | 3.5 | Grs2-pending | Golgi reassembly stacking |
| | | | protein 2 |
| W08453 | 1.9 | Dsn | Destrin |
| AA472795 | 1.9 | Slc6a6 | Retinal taurine transporter; |
| | | | solute carrier family 6, |
| | | | member 6 |
| W34349 | 2.0 | F12 | Coagulation factor XII |
| | | | (Hageman factor) |
| W17473 | −2.4 | Agt | Angiotensinogen |
| W12913 | −2.2 | Hamp | Hepcidin antimicrobial peptide |

Unknown function

| | | | |
|---|---|---|---|
| W85397 | 2.0 | | mf48c06.rl Soares mouse |
| | | | embryo NbME13.5 14.5 |
| | | | Mus musculus cDNA clone |
| | | | IMAGE:408298 |
| AA250708 | −2.5 | A1266885 | Expressed sequence A1266885 |
| C79645 | 1.8 | C79645 | Expressed sequence C79645 |
| AA000467 | 2.0 | 1110002E23Rik | RIKEN cDNA |
| | | | 1110002E23 gene |
| AA066610 | 1.9 | 1810034D23Rik | RIKIEN cDNA |
| | | | 1810034D23 gene |
| AA260798 | 1.9 | 2310009N05Rik | RIKEN cDNA |
| | | | 2310009NO5 gene |
| AA285530 | 1.9 | | vb90b12.rl Soares mouse |
| | | | 3NbMS Mus musculus |
| | | | cDNA clone IMAGE:764255 5′, |
| | | | mRNA sequence._ |
| AA538556 | 2.0 | 1810020N21Rik | RIKEN cDNA |
| | | | 1810020N21 gene |
| AA592283 | 1.8 | AA960392 | Expressed sequence AA960392 |
| AA655730 | 1.8 | 2700059D21Rik | RIKEN cDNA |
| | | | 2700059D21 gene |
| AA666984 | 2.0 | | vq87g01.r1 Knowles Solter |
| | | | mouse blastocyst B3 |
| | | | Mus musculus cDNA clone |
| | | | IMAGE: 1109328 5′, |
| | | | mRNA sequence. |
| AA038437 | 2.6 | 0610008L05Rik | Similar to eukaryotic |
| | | | translation initiation factor 4 |
| | | | gamma, 1 |
| AA124572 | 1.9 | | mp72b03.rl Soares_{—} |
| | | | thymus_2NbMT Mus |
| | | | musculus eDNA clone |
| | | | IMAGE:574733 5′ similar |
| | | | to gb:X52851_rnal |
| | | | PEPTIDYL-PROLYL CIS- |
| | | | TRANS ISOMERASE A |
| | | | (HUMAN); gb:X52803 |
| | | | Mouse mRNA for cyclophilin |
| | | | (MOUSE);, mRNA |
| | | | sequence. |
| W12899 | −2.9 | | mb19d12.rl Soares mouse |
| | | | p3NMF19.5 Mus |
| | | | musculus eDNA clone |
| | | | IMAGE:329879 5′ similar |
| | | | to gb:X13839 ACTIN, |
| | | | AORTIC SMOOTH |
| | | | MUSCLE (HUMAN);, |
| | | | mRNA sequence. |
| W21013 | −2.6 | 1110061M03Rik | RIKEN cDNA |
| | | | 1110061M03 gene |
| W12941 | 2.7 | 1110004C05Rik | RIKEN cDNA |
| | | | 1110004C05 gene |
| W3021 1 | 2.1 | | mc25d02.rl Soares mouse |
| | | | p3NMF19.5 Mus |
| | | | musculus eDNA clone |
| | | | IMAGE:349539 5′, mRNA |
| | | | sequence. |
| W64827 | 2.4 | 1810030E05Rik | RIKEN cDNA |
| | | | 1810030E05 gene |
| C77421 | 2.5 | | C77421 Mouse 3.5-dpc |
| | | | blastocyst cDNA Mus |
| | | | musculus cDNA clone |
| | | | J0030G04 3′ similar to |
| | | | Mouse B10.VL30LTR gene, |
| | | | 5′ flank, mRNA |
| | | | sequence. |
| W30137 | 1.9 | 2410038A03Rik | RIKEN cDNA |
| | | | 2410038A03 gene |

In certain embodiments, the animals have type I diabetes; in other embodiments, the animals have type II diabetes. This invention also provides databases of these diabetes-associated sequences and proteins and antibodies to the diabetes proteins.

The above-described diabetes polynucleotides and polypeptides, databases, and antibodies have both diagnostic and therapeutic uses. In one aspect, this invention provides methods for diagnosis of diabetes by monitoring the expression levels of such genes. In another aspect, the invention provides methods of modulating the expression levels of such genes to treat diabetes and diabetic complications. The invention also provides methods of modulating the activity of the proteins encoded by these genes to treat diabetes. In yet another aspect, the invention provides a screening assay that identifies compounds which modulate expression levels of the genes that are over or under expressed in diabetes or compounds that modulate the activity levels of polypeptides expressed by such genes. In certain instances, these compounds can also be used to treat diabetes and diabetic complications.

These embodiments are described in the following sections.

II. Identification of Diabetes-associated Sequences

1. Characteristics of the Sequences

The present invention provides nucleic acid and protein sequences that are differentially expressed in diabetes, herein termed “diabetes sequences.” Diabetes sequences can include both nucleic acid and amino acid sequences. The nucleic acid sequences can be full length genes or ESTs. As outlined below, diabetes sequences include those that are up-regulated (i.e., expressed at a higher level) in diabetes, as well as those that are down-regulated (i.e., expressed at a lower level).

In one embodiment, diabetes sequences are those that are up-regulated in diabetic animals or humans; that is, the expression of these genes is higher in the tissue from diabetic organisms as compared to tissue from non-diabetic organisms (see, e.g., Table 1). “Up-regulation” as used herein means, when the ratio is presented as a number greater than one, that the ratio is greater than one, 1.5 or greater, or 2.0 or greater.

In another embodiment, diabetic sequences are those that are down-regulated in diabetes; that is, the expression of these genes is lower in tissue from diabetic organisms as compared to tissue from non-diabetic organisms (see, e.g., Table 1). “Down-regulation” as used herein means, when the ratio is presented as a number greater than one, that the ratio is greater than one, 1.5 or greater, or 2.0 or greater, or, when the ratio is presented as a number less than one, that the ratio is less than one, 0.5 or less, 0.25 or less.

Diabetes sequences can be obtained from humans, as well as other animals, including vertebrates, including mammals, including rodents (rats, mice, hamsters, guinea pigs, etc.), primates, farm animals (including sheep, goats, pigs, cows, horses, etc.) and pets (dogs, cats, etc.). These sequences are useful in animal models of disease and drug evaluation. Diabetes sequences from other organisms can also be obtained using the techniques outlined below.

2. Methods of Identifying Sequences

Expression Profiles

In certain embodiments, diabetes-associated sequences are identified using expression profiles. An expression profile of a particular sample is essentially a “fingerprint” of the state of the sample; while two states may have any particular gene similarly expressed, the evaluation of a number of genes simultaneously allows the generation of a gene expression profile that is characteristic of diabetes. By comparing expression profiles of tissue in non-diabetic vs. diabetic humans and animals, information regarding which genes are important (including both up- and down-regulation of genes) in diabetes is obtained. In addition, by comparing gene expression levels for different cellular states in the diabetes phenotype (i.e., different severities of diabetes), diabetes sequences associated with a particular degree of severity of diabetes can be identified.

Expression profiles can be generated for that population of genes in any organ in the body that can be affected by diabetes. In one embodiment, expression profiles are generated for genes expressed in the liver. The microarrays can contain probe sets with fragments of genes, full-length genes, cDNAs, mRNA, ESTs, etc. In certain embodiments, the microarrays contain probe sets with both murine genes and expressed sequence tags. Suitable biochips are commercially available, e.g., from Affymetrix.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus diabetes tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection. As outlined above, the change in expression (i.e., upregulation or downregulation) is typically at least about 50%, at least about 100%, at least about 150%, at least about 200%, or from 300 to at least 1000%.

Identification via Homology or Linkage

Additional diabetes-associated sequences can be identified by substantial nucleic acid and/or amino acid sequence homology or linkage to the diabetes-associated sequences outlined herein. Such homology can be based upon the overall nucleic acid or amino acid sequence, and is generally determined as outlined below, using either homology programs or hybridization conditions.

The diabetic nucleic acid sequences of the invention, e.g., the sequences in Table 1, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the diabetes genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, http://www.ncbi.nlm.nih.gov/unigene/).

Once the diabetes nucleic acid is identified, it can be cloned and, if necessary, its constituent parts recombined to form the entire diabetes nucleic acid coding regions or the entire mRNA sequence. Once isolated from its natural source, e.g., contained within a plasmid or other vector or excised therefrom as a linear nucleic acid segment, the recombinant diabetes nucleic acid can be further used as a probe to identify and isolate other diabetes nucleic acids, e.g., extended coding regions. It can also be used as a “precursor” nucleic acid to make modified or variant diabetes nucleic acids and proteins.

III. Applications of Diabetes Associated Sequences

A. Overview

The diabetes sequences of this invention have multiple uses. One of skill will readily appreciate that particular use of the sequence will depend on the role that the particular sequence and the encoded polypeptide play in diabetes—is it a marker for disease, a contributing factor, or a combination thereof?

Certain diabetes associated sequences are over- or under-expressed in diabetic organisms as a consequence of the primary causes of diabetes (lack of insulin secretion, defective insulin signaling, etc.). These sequences can be used for diagnostic/prognostic applications and screening applications, e.g., biochips comprising nucleic acid probes or PCR microtiter plates with selected probes to the diabetes sequences. In certain embodiments, the diabetes sequences can be used to evaluate a particular treatment regime; does a drug act to treat the symptoms and complications of diabetes in a particular patient. Similarly, diagnosis and treatment outcomes may be done or confirmed by comparing patient samples with the known expression profiles. Furthermore, these gene expression profiles (or individual genes) allow screening of drug candidates with an eye to mimicking or altering a particular expression profile; e.g., screening can be done for drugs that suppress the diabetes expression profile. This may be done by making biochips comprising sets of the important genes expressed in diabetes, which can then be used in these screens. PCR methods may be applied with selected primer pairs, and analysis may be of RNA or of genomic sequences. These methods can also be done on a protein basis; that is, protein expression levels of the diabetes-modulated proteins can be evaluated for diagnostic purposes or to screen candidate therapeutic agents.

In other instances, certain genes that are overexpressed in response to a primary cause of diabetes are indicative of protective responses activated by the cell. In some cases, it may be desirable to further upregulate the expression of such genes.

Other genes that are over- or under-expressed in diabetic organisms induce the disease by interfering with the secretion of insulin, physiology of islet cells, the insulin signalling pathway, or some other pathway or process that affects glucose homeostasis. Thus diabetes and diabetic complications can be treated by modulating the gene expression level and/or protein expression level of these genes to normal levels or modulating the activity of the polypeptides encoded by these genes. In certain embodiments, diabetes-modulated nucleic acid sequences are administered for gene therapy purposes, including the administration of antisense nucleic acids. Alternatively, diabetes-induced proteins (including antibodies and other modulators thereof) are administered as therapeutic drugs or as protein or DNA vaccines. It will be recognized by one of skill in the art that detection of the expression level of such genes can also be used for diagnostic/prognostic purposes.

In still other instances, certain diabetes-associated sequences influence aspects of subject's physiology, e.g., drug metabolism, that should considered when managing diabetes or other aspects of the subject's health. For example, a better understanding of the alterations in drug metabolism during diabetes can help ameliorate unwanted side effects from the multitude of drugs used to control diabetes.

B. Functions of Genes in Table 1

Among the sequences provided by this invention are the 47 sequences described in Table-I. These differentially expressed genes can be grouped into categories based on their putative functions as reported in scientific literature. As described in the above section, these putative functions can be used to determine whether a particular gene is best used for diagnostic, therapeutic, or disease management purposes.

Metaholism. Thirty eight percent of the genes in Table 1 are linked to the metabolism of carbohydrate, fat, or protein. Streptozotocin-induced diabetes (SID) increases the expression of lactate dehydrogenase 1, which catalyzes the interconversion of pyruvate and lactate. Increased hepatic expression of this isozyme suggests that glycolysis has increased importance in energy production in the liver of SID mice. This interpretation is consistent with the elevated hepatic lactate levels in streptozotocin (STZ) treated rats (Kondoh et al., Res. Exp. Med. (Berl) 192:407-414 (1992)). SID decreases the expression of two members of the alcohol dehydrogenase superfamily, alcohol dehydrogenase 1 and sorbitol dehydrogenase 1, consistent with the decrease in the activities of these enzymes in diabetic rats (Lakshman et al., Alcohol Clin. Exp. Res. 12:407-411 (1988); Hoshi et al., Biochem. J. 318(Pt 1):119-123 (1996)). Sorbitol dehydrogenase 1 is a major contributor to diabetic complications (J. H. Kinoshita, Exp. Eye Res. 50:567-573 (1990)).

Glucocorticoids are important inducers of gluconeogenesis (Granner et al., J. Biol. Chem. 265:10173-10176 (1990)). Diabetes is accompanied by elevated serum glucocorticoid levels. Decreased expression of corticosteroid binding globulin in SID mice can increase the levels of bioavailable corticosterone, further exacerbating SID-related hyperglycemia.

SID increases expression of the gene for apolipoprotein H. This result is consistent with the elevation of serum apolipoprotein H found in diabetes and dyslipidemia (Crook et al., Ann. Clin Biochem. 38:494-498 (2001). The physiological function of this apolipoprotein is unclear. It appears to be involved in lipid metabolism, haemostasis, and binding of some groups of antibodies to anionic phospholipids (McNeil et al., Proc. Natl. Acad. Sci. U.S. A 87:4120-4124 (1990).

SID increases the expression of genes encoding mitochondrial proteins. Cytochrome c oxidase and NADH dehydrogenase 1 are components of the mitochondrial electron transport system which were increased in SID. Increased expression of these genes can contribute to the increase in mitochondrial respiration observed in uncontrolled diabetes and STZ-treated rats (Antonetti et al., J Clin Invest 95:1383-1388 (1995); Mackerer et al., Proc. Soc. Exp. Biol. Med. 137:992-995 (1971)). SID increases the expression of genes associated with protein degradation. A common feature of insulin deficiency is increased protein catabolism in muscle and other tissues (Smith et al., Diabetes 38:1117-1122 (1989)). The expression-of two-key enzymes of the urea-cycle, arginase 1 and argininosuccinate synthetase 1, is increased (Table 1), in accord with previous reports (W. E. Duncan and J. S. Bond, Am. J. Physiol 241:E151-E159 (1981); Jorda et al., Enzyme 26:240-244 (1981)). Nitrogen derived from elevated amino acid catabolism is disposed of via the urea cycle. The increased protein degradation associated with insulin insufficiency also extends to the liver (Hutson et al., Proc. Natl. Acad. Sci. U.S. A. 79:1737-1741 (1982); Garlick et al., Acta Biol. Med. Ger 40:1301-1307 (1981)). Increased expression of hepatic histidine ammonia lyase, which catalyzes the deamination of histidine (a gluconeogenic amino acid), and a proteasome subunit (Table 1) are consistent with enhanced protein degradation. Together, these results suggest SID promotes proteolysis, perhaps via the proteasome pathway.

Stress Response and Xenobiotic Metabolism. Thirty six percent of the genes in Table 1 encode cytoprotective stress proteins or enzymes responsible for the metabolism of xenobiotics. mRNA for heat shock 70 kD protein 5, hypoxia inducible factor 1 and prolyl 4-hydroxylase, beta polypeptide are induced. The cytoprotective functions of these genes include the repair or degradation of proteins damaged by glycoxidation (Medina et al., Biochem. J. 307 (Pt 3):631-637 (1995); M. Y. Sherman and A. L., EXS 77:57-78 (1996)). Hyperglycemia produces free radicals by promoting glycation and oxidation reactions between reducing sugars and proteins (I. C. West, Diabet. Med. 17:171-180 (2000)). The resulting oxidative stress is amplified by a cycle of metabolic stress and tissue damage, which leads to additional free radical production. Oxidative stress is thought to play an important role in the development of the complications of diabetes.

SID differentially affects the expression of genes for phase I and II enzymes. Glutathione S-transferase μ2 and π2 isozymes increased with SID. Glutathione S-transferases detoxify electrophilic xenobiotics by conjugating them with glutathione (Coles et al., Crit Rev. Biochem. Mol. Biol. 25:47-70 (1990)). SID has mixed effects on the expression of cytochrome P450's. It increases expression of the flavin-containing monooxygenase 5 gene and two cytochrome P450, 3a subfamily members. These enzymes metabolize numerous drugs and endogenous substances in the liver. In contrast, SID decreases the expression of other xenobiotic-metabolizing enzymes including thioether S-methyltransferase and the cytochrome P450, 1a2, 2b13, and 2c29 genes. These results are consistent with the previously reported differential modulation of P450 isoform expression (Bellward et al., Mol. Pharmacol. 33:140-143 (1988); Shimojo et al., Biochem. Pharmacol. 46:621-627 (1993); Barnett et al., Biochem. Pharmacol. 40:393-397 (1990)). While the mechanisms underlying this differential regulation are largely unknown, the expression of the P450, 2e1 isozyme in spontaneously and chemically induced diabetes has been linked to elevated levels of plasma ketone bodies (Bellward et al., Mol. Pharmacol. 33:140-143 (1988); Dong et al., Arch. Biochem. Biophys. 263:29-35 (1988)).

Growth Factors and Signal Transducers. SID increases the expression of hepatic mRNA for insulin-like growth factor binding protein 2. This result is consistent with previous reports of increased mRNA and serum protein levels of insulin-like growth factor binding protein 2 in SID rats (Ooi et al., Biochem. Biophys. Res. Commun. 189:1031-1037 (1992); Rodgers et al., Proc. Soc. Exp. Biol. Med. 210:234-241 (1995)). Increased serum levels of insulin-like growth factor binding proteins result in decreased bioavailability of IGF-I, and thus reduced cell division and growth. SID also increases RAB 1, a Ras-related protein which regulates the earliest stage of protein trafficking through the secretory pathway (Schimmoller et al., J. Biol. Chem. 273:22161-22164 (1998)). Its function is critically important for all types of cellular growth. RAB1 overexpression is linked to modified protein trafficking leading to altered cell structure and function (Wu et al., Circ. Res. 89:1130-1137 (2001)).

Immune and Inflammation Response. SID alters the expression of 3 genes in Table 1 linked to the immune or inflammatory responses. SID decreases the expression of complement component 4. Reduced complement component 4 expression in SID mice is consistent with the reduced level and rate of synthesis of this protein in type 1 diabetic humans (Charlesworth et al., Diabetologia 30:372-379 (1987)). SID increases the expression of inter-alpha trypsin inhibitor, heavy chain 3, which is a serum protease inhibitor. Uncontrolled protease activity may act in concert with the ubiquitin-proteasome proteolytic pathway to enhance muscle atrophy in diabetic animals (Price et al., J. Clin. Invest 98:1703-1708 (1996)).

IV. Database of Diabetes-Associated Proteins

The diabetes associated proteins of this invention can collectively provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients with diabetes.

Thus, the present invention provides a database that includes at least one set of assay data. The data contained in the database is acquired, e.g., using array analysis either singly or in a library format. The database can be in substantially any form in which data can be maintained and transmitted, but is typically an electronic database. The electronic database of the invention can be maintained on any electronic device allowing for the storage of and access to the database, such as a personal computer, but is typically distributed on a wide area network, such as the World Wide Web.

The compositions and methods for identifying and/or quantitating the relative and/or absolute abundance of a variety of molecular and macromolecular species from a biological sample undergoing diabetes, i.e., the identification of diabetes-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, typically, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be-viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioinformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinformatics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., liver biopsy or another tissue specimen from a patient with diabetes. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention typically provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In one embodiment, the invention provides a computer system for comparing a query target to a database containing an array of data structures, such as an assay result obtained by the method of the invention, and ranking database targets based on the degree of identity and gap weight to the target data. A central processor can be initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

V. Antibodies against Diabetes Sequences for Diagnostic and Therapeutic Purposes

In certain embodiments, this invention provides antibodies to the above-described diabetes sequences for immunotherapy or immunodiagnosis. For example, administration of antibodies can be used to reduce the levels of overexpressed genes that contribute to diabetes or antibodies can be used to quantitate expression levels for diagnostic purposes.

A. Production of Antibodies

When a diabetes protein is to be used to generate antibodies, the diabetes protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller diabetes protein will be able to bind to the full-length protein, particularly linear epitopes. Typically, the epitope is unique; that is, antibodies generated to a unique epitope show little or no cross-reactivity.

The diabetes proteins can be coupled, using standard technology, to affinity chromatography columns. These columns may then be used to purify diabetes antibodies useful for production, diagnostic, or therapeutic purposes.

The diabetes antibodies of the invention specifically bind to diabetes proteins. By “specifically bind” herein is meant that the antibodies bind to the protein with a Kd of at least about 0.1 mM, more usually at least about 1 μM, at least about 0.1 μM or better, and 0.01 μM or better. Selectivity of binding is also important.

In certain embodiments, the diabetes protein against which the antibodies are raised is an intracellular protein. In this case, the antibody may be conjugated to a protein or other entity which facilitates entry into the cell. In one case, the antibody enters the cell by endocytosis. In another embodiment, a nucleic acid encoding the antibody is administered to the individual or cell. Moreover, wherein the diabetes protein can be targeted within a cell, i.e., the nucleus, an antibody thereto contains a signal for that target localization, i.e., a nuclear localization signal.

Methods of Preparing Polyclonal Monoclonal, & Humanized Antibodies

Methods of preparing polyclonal antibodies are well known (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of Table 1 or fragment thereof or a fusion protein thereof. It may be useful to conjugate the immunizing agent to a protein known to be immunogenic in the mammal being immunized. Immunogenic proteins include, e.g., keyhole limpet hemocyanin, serum albumin, bovine thyroglobulin, and soybean trypsin inhibitor. Adjuvants include, e.g., Freund's complete adjuvant and MPL-TDM adjuvant (monophosphoryl Lipid A, synthetic trehalose dicorynomycolate). The immunization protocol may be selected by one skilled in the art.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Table 1, or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and primate origin. Usually, rat or mouse myeloma cell lines are employed. The hybridoma cells may be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are typically monoclonal, typically human or humanized, antibodies that have binding specificities for at least two different antigens or that have binding specificities for two epitopes on the same antigen. In one embodiment, one of the binding specificities is for a protein encoded by a nucleic acid of Table 1 or a fragment thereof, the other one is for any other antigen, and for a cell-surface protein or receptor or receptor subunit, one that is specific to cells that altered in diabetes. Alternatively, tetramer-type technology may create multivalent reagents.

In one embodiment the antibodies to the diabetes proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human-like antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in virtually all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

B. Antibody Uses

Immunotherapy

By immunotherapy is meant the treatment of diabetes with an antibody raised against a diabetes proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. The antigen may be provided by injecting a polypeptide against which antibodies are desired to be raised into a recipient, or contacting the recipient with a nucleic acid capable of expressing the antigen and under conditions for expression of the antigen, leading to an immune response.

In certain embodiments, the antibodies to diabetes protein are capable of reducing the biological function of a diabetes protein, as is described below. That is, the addition of anti-diabetes protein antibodies (either polyclonal or monoclonal) may reduce or eliminate the diabetes. Generally, the decrease in activity is at least 25%, at least 50%, or at least 95-100%.

In one embodiment the diabetes proteins against which antibodies are raised are secreted proteins. Without being bound by theory, it is believed that antibodies used for immunotherapy bind and prevent increased levels of the secreted protein from interfering with insulin receptor signalling, insulin secretion, etc.

In another embodiment, the diabetes protein to which antibodies are raised is a transmembrane protein. Without being bound by theory, it is believed that antibodies used for this treatment can bind the extracellular domain of the diabetes protein and prevent it from binding to other proteins, such as circulating ligands or cell-associated molecules. The antibody may cause down-regulation of the transmembrane diabetes protein. The antibody may be a competitive, non-competitive or uncompetitive inhibitor of protein binding to the extracellular domain of the diabetes protein. The antibody may be an antagonist of the diabetes protein or may prevent activation of the transmembrane diabetes protein. In some instances the antibody belongs to a sub-type that activates serum complement when complexed with the transmembrane protein thereby mediating cytotoxicity or antigen-dependent cytotoxicity (ADCC). Thus, diabetes is treated by administering to a patient antibodies directed against the transmembrane diabetes protein. Antibody-labeling may activate a co-toxin or localize a toxin payload.

In another embodiment, the antibody is conjugated to an effector moiety. The effector moiety can be any number of molecules, including labeling moieties such as radioactive labels or fluorescent labels, or can be a therapeutic moiety. In one aspect the therapeutic moiety is a small molecule that modulates the activity of the diabetes protein. In another aspect the therapeutic moiety modulates the activity of molecules associated with or in close proximity to the diabetes protein.

Diagnostic Uses

In another embodiment, antibodies find use in diagnosing diabetes from blood, serum, plasma, stool, and other samples. Such samples, therefore, are useful as samples to be probed or tested for the presence of diabetes proteins. Antibodies can be used to detect a diabetes protein by previously described immunoassay techniques including ELISA, immunoblotting (western blotting), immunoprecipitation, BIACORE technology and the like.

VI. Methods for Assaying Diabetes-Associated Gene and Protein Expression Levels for Diagnostic and Prognostic Purposes

In some embodiments, the diabetes proteins, antibodies, nucleic acids, modified proteins and cells containing diabetes sequences are used in diagnostic assays. This can be performed on an individual gene or corresponding polypeptide level. In one embodiment, expression profiles are used, usually in conjunction with high throughput screening techniques (i.e., biochips) to allow monitoring of the expression of diabetes genes and/or corresponding polypeptides.

In another embodiment, the diabetes proteins, antibodies, nucleic acids, modified proteins and cells containing diabetes sequences are used in prognosis assays to determine the severity of diabetes. As above, gene expression profiles can be generated that correlate to diabetes, in terms of the severity of the disease. Again, this may be done on either a protein or gene level, typically with genes using biochips for the detection and quantification of diabetes sequences in a tissue or patient. The assays proceed as outlined above for diagnosis.

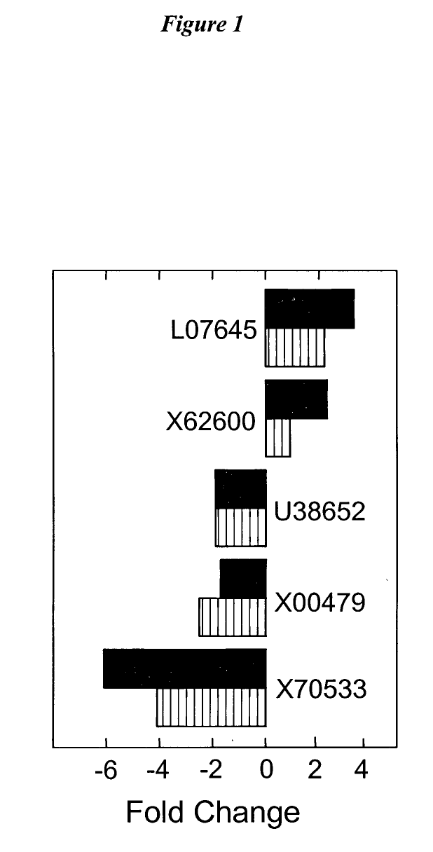

The genes used as diagnostic markers can be any gene found to be either overexpressed or underexpressed in diabetic subjects. In one embodiment, the genes are those found in Table 1 and FIG. 1, e.g., genes encoding lactate dehydrogenase 1, the alcohol dehydrogenase superfamily, alcohol dehydrogenase 1, apolipoprotein H, cytochome c oxidase, NADH dehydrogenase 1, arginase 1, arginosuccinate synthetase 1, glutathione S-transferases, cytochrome P450s, insulin-like growth factor binding protein 2, or complement component 4.

Methods for analysis of the RNA and protein expression levels of diabetes-associated sequences and protein are described below.

A. Methods for Assaying Gene Expression

In some embodiments, the expression levels of multiple diabetes-associated genes in the livers of diabetic and non-diabetic animals are assayed using high-throughput technology for diagnostic and prognostic purposes.

Gene expression monitoring can be performed on a single polynucleotide or simultaneously for a number of polynucleotides. In one embodiment, unlabeled probes for diabetes nucleic acids are attached to biochips and a sample containing labeled target nucleic acid is applied to the chip. In another embodiment, the unlabeled nucleic acid to be examined (target nucleic acid) is attached to a solid support and labeled probe is used to detect a diabetes nucleic acid. PCR techniques for measurement of gene expression levels can be used to provide greater sensitivity.

Although DNA or RNA encoding the diabetes protein may be detected, of particular interest are methods wherein an mRNA encoding a diabetes protein is detected. Probes to detect mRNA can be a nucleotide/deoxynucleotide probe that is complementary to and hybridizes with the mRNA and includes, but is not limited to, oligonucleotides, cDNA or RNA.

1. Detection of Labeled Probe bound to Immobilized Target Nucleic Acid

In one embodiment, mRNA is detected after immobilizing the nucleic acid to be examined on a solid support such as nylon membranes and hybridizing a labeled probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the-target mRNA. Following washing to remove the non-specifically bound probe, the label is detected. For example, a digoxygenin labeled riboprobe (RNA probe) that is complementary to the mRNA encoding a diabetes protein is detected by binding the digoxygenin with an anti-digoxygenin secondary antibody and developed with nitro blue tetrazolium and 5-bromo-4-chloro-3-indoyl phosphate.

Preparation of Samples Containing the Unlabeled Target Sequence

Samples containing target sequence can be analyzed as follows. For example, the sample may be treated to lyse the cells, using known lysis buffers, electroporation, etc., with purification and/or amplification such as PCR performed as appropriate. For example, an in vitro transcription reaction is performed.

Nucleic Acid Probes

In one embodiment, nucleic acid probes to diabetes nucleic acids (both the nucleic acid sequences outlined in the figures and/or the complements thereof) are made. The nucleic acid probes are designed to be substantially complementary to the diabetes nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under appropriate reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, from about 10 to about 80 bases, or from about 30 to about 50 bases. That is, generally complements of ORFs or whole genes are not used. In some embodiments, nucleic acids of lengths up to hundreds of bases can be used.

In some embodiments, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

Labeled Probe

In some embodiments, the probe is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis. Generally, the nucleic acids are labeled with biotin-FITC or PE, or with cy3 or cy5.

Attachment of the Target Nucleic Acids to the Solid Support

As will be appreciated by those in the art, nucleic acids can be attached or immobilized to a solid support in a wide variety of ways. By “immobilized” and grammatical equivalents herein is meant the association or binding between the target nucleic acid and the solid support is sufficient to be stable under the conditions of binding, washing, analysis, and removal as outlined below. The binding can typically be covalent or non-covalent. By “non-covalent binding” and grammatical equivalents herein is typically meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to a biochip in a wide variety of ways, as will be appreciated by those in the art. As described herein, the nucleic acids can either be synthesized first, with subsequent attachment to the biochip, or can be directly synthesized on the biochip.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In one embodiment, the nucleic acids can be synthesized in situ, using well known photolithographic techniques, such as those described in-WO 95/25116; WO 95/35505; U.S. Pat. Nos. 5,700,637 and 5,445,934; and references cited within, all of which are expressly incorporated by reference; these methods of attachment form the basis of the Affymetrix GeneChip™ technology.

Biochips

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. One such substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In one embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups. Using these functional groups, the probes can be attached using functional groups on the probes. For example, nucleic acids containing amino groups can be attached to surfaces comprising amino groups, e.g., using linkers as are known in the art; e.g., homo-or hetero-bifunctional linkers as are well known (see, 1994 Pierce Chemical Company catalog, technical section on cross-linkers, pages 155-200). In addition, in some cases, additional linkers, such as alkyl groups (including substituted and heteroalkyl groups) may be used.

Hybridization and Sandwich Assays

Nucleic acid assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, attached to a solid support, and then the labeled probe is added under conditions that allow the formation of a hybridization complex.

A variety of hybridization conditions may be used in the present invention, including high, moderate and low stringency conditions as outlined above. The assays are generally run under stringency conditions which allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with certain embodiments outlined below. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

2. Detection of Labeled Target Nucleic Acid Bound to Immobilized Probe

One of skill will readily appreciate that methods similar to those in the preceding section can be used in embodiments where the a nucleic acid to be examined is attached to a solid support and labeled probe is used to detect the diabetes nucleic acid.

3. Amplification-based Assays

Amplification-based assays can also be used measure the expression level of diabetes-associated sequences for diagnostic and prognostic purposes. These assays are typically performed in conjunction with reverse transcription. In such assays, a diabetes-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of diabetes-associated RNA. Methods of quantitative amplification are well known to those of skill in the art. Detailed protocols for quantitative PCR are provided, e.g., in Innis et al., PCR Protocols, A Guide to Methods and Applications (1990).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

B. Methods of Assaying Protein Expression Levels

The expression levels of multiple proteins can also be performed. Similarly, these assays may also be performed on an individual basis.

As described and defined herein, diabetes proteins, including intracellular, transmembrane or secreted proteins, find use as markers of diabetes. Detection of these proteins in putative diabetes tissue allows for detection or diagnosis of diabetes. In one embodiment, antibodies such as those described in Section V of the specification are used to detect diabetes proteins. One method separates proteins from a sample by electrophoresis on a gel (typically a denaturing and reducing protein gel, but may be another type of gel, including isoelectric focusing gels and the like). Following separation of proteins, the diabetes protein is detected, e.g., by immunoblotting with antibodies raised against the diabetes protein. Methods of immunoblotting are well known to those of ordinary skill in the art.

In another method, antibodies to the diabetes protein find use in in situ imaging techniques for detection of diabetes protein(s), e.g., in histology (e.g., Methods in Cell Biology: Antibodies in Cell Biology, volume 37 (Asai, ed. 1993)). In this method cells are contacted with from one to many antibodies to the diabetes protein(s). Following washing to remove non-specific antibody binding, the presence of the antibody or antibodies is detected. In one embodiment, the antibody is detected by incubating with a secondary antibody that contains a detectable label, e.g., multicolor fluorescence or confocal imaging. In another method the primary antibody to the diabetes protein(s) contains a detectable label, e.g., an enzyme marker that can act on a substrate. In another embodiment each one of multiple primary antibodies contains a distinct and detectable label. This method finds particular use in simultaneous screening for a plurality of diabetes proteins. Many other histological imaging techniques are also provided by the invention.

In one embodiment the label is detected in a fluorometer which has the ability to detect and distinguish emissions of different wavelengths. In addition, a fluorescence activated cell sorter (FACS) can be used in the method.

VII. Methods of Modulating Gene Expression Levels for Therapeutic Purposes

In one aspect, the invention provides methods of treating diabetes or diabetic complications by modulating the expression level of diabetes-associated sequences of this invention. The specific therapeutic effect will depend on the nature of the diabetes-associated sequence (i.e., which organs the sequences are expressed in, the predicted or known function of the polypeptide encoded by the sequence). The methods can be used to treat diabetes or any diabetic complications, including, but not limited to, alterations in xenobiotic metabolism, retinopathy, nephropathy, neuropathy, or hepatic functional abnormalities. In some embodiments, the diabetes-associated sequences are the genes described in Table 1 and FIG. 1.

In one embodiment, the expression level of sorbitol dehydrogenase 1 is upregulated to normal levels to treat diabetic complications (retinopathy and neuropathy). In another embodiment, the expression level of the corticosteroid binding protein is upregulated to normal levels in order to reduce corticosterone levels to normal levels. Excess corticosterone induces gluconeogenesis, which exacerbates diabetes. In yet another embodiment, genes encoding cytoprotective stress proteins (i.e., heat shock 70 kD protein 5, hypoxia inducible factor 1, prolyl 4-hydroxylase beta polypeptide, etc.) are upregulated to reduce hyperglycemia-induced oxidative stress, which is thought to play an important role in the development of diabetic complications. In another embodiment, levels of RAB 1 expression are downregulated to normal levels to prevent modified protein trafficking patterns that can lead to altered cellular structure and function. In another embodiment, expression of inter-alpha trypsin inhibitor (heavy chain 3) is upregulated to control protease activity, which is postulated to enhance muscle atrophy in diabetic subjects.

It will be appreciated by those of skill in the art that the modulation will either comprise reducing or increasing the expression level of a gene, depending on the change in expression levels associated with diabetes. For example, when the diabetes sequence is down-regulated in diabetes, such state may be reversed by increasing the amount of diabetes gene product in the cell. This can be accomplished, e.g., by overexpressing the endogenous diabetes gene or administering a gene encoding the diabetes sequence, using known gene-therapy techniques. Alternatively, e.g., when the diabetes sequence is up-regulated in diabetes, the activity of the endogenous diabetes gene is decreased, e.g., by the administration of a diabetes antisense nucleic acid.

Gene expression levels can be modulated using any method known to those of skill in the art. Typically, expression levels are modulated using anti-sense polynucleotides, ribozymes, or small interfering RNAs.

Antisense Polynucleotides

In certain embodiments, the activity of a diabetes-associated protein is downregulated, or entirely inhibited, by the use of antisense polynucleotide, i.e., a nucleic acid complementary to, and which can hybridize specifically to, a coding mRNA nucleic acid sequence, e.g., a diabetes protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the diabetes protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for diabetes sequences. Antisense or sense oligonucleotides, according to the present invention, comprise a fragment generally at least about 14 nucleotides or from about 14 to 30 nucleotides. The ability to derive an antisense or a sense oligonucleotide, based upon a cDNA sequence encoding a given protein is described in, e.g., Stein & Cohen Cancer Res. 48:2659 (1988) and van der Krol et al. BioTechniques 6:958 (1988)).

Polynucleotide modulators, e.g., anti-sense DNA, of diabetes may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Conjugation of the ligand binding molecule should not substantially interfere with the ability of the ligand binding molecule to bind to its corresponding molecule or receptor, or block entry of the sense or antisense oligonucleotide or its conjugated version into the cell. Alternatively, a polynucleotide modulator of diabetes may be introduced into a cell containing the target nucleic acid sequence, e.g., by formation of an polynucleotide-lipid complex, as described in WO 90/10448.

Ribozymes

In addition to antisense polynucleotides, ribozymes can be used to target and inhibit transcription of diabetes-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (See, e.g., Castanotto et al., Adv. in Pharmacology 25: 289-317 (1994) for a general review of the properties of different ribozymes).

The general features of hairpin ribozymes are described, e.g., in Hampel et al., Nucl. Acids Res. 18:29-9-304 (1990); European Patent Publication No. 0 360 257; U.S. Pat. No. 5,254,678. Methods of preparing are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

VIII. Methods of Modulating the Activity of Diabetes-Associated Proteins for Therapeutic Purposes

In other aspects, this invention provides methods of modulating the activity of diabetes-associated proteins of this invention. The specific therapeutic effect will depend on the nature of the diabetes-associated sequence (i.e., which organs the sequences are expressed in, the predicted or known function of the polypeptide encoded by the sequence). The methods can be used to treat diabetes or any diabetic complications, including, but not limited to, alterations in xenobiotic metabolism, retinopathy, nephropathy, neuropathy, or hepatic functional abnormalities. Typical hepatic functional abnormalities include loss of hepatic insulin sensitivity; excessive protein breakdown; reduction of protein synthesis; excessive hepatic glucose production (gluconeogenesis); reduction in cell growth and regeneration; excessive cell death (apoptosis); serum dyslipidemia resulting from altered hepatic lipid and protein metabolism; altered drug, metabolite, and xenobiotic metabolism; enhanced oxidative damage and cellular stress; and enhanced inflammatory and disregulated immune responsiveness. In some embodiments, the diabetes-associated sequences are the genes described in Table 1 and FIG. 1. It will be readily appreciated by one of skill of art that the genes described in Section VII can also be manipulated by modulating activity levels.

It will be appreciated by those of skill in the art that the modulation will either comprise reducing or increasing the activity level of a diabetes protein, depending on the change in expression levels associated with diabetes. For example, when the diabetes sequence is down-regulated in diabetes, such state may be reversed by increasing the activity of the diabetes gene product in the cell. This can be accomplished using, e.g., a small molecule activator. Alternatively, e.g., when the diabetes sequence is up-regulated in diabetes, the activity of the endogenous diabetes protein is decreased, e.g., by the administration of a diabetes inhibitors.

Small molecule inhibitors and activators can be identified using methods described in the following section.

IX. Methods of Screening for Drugs

A. Introduction

The present invention provides novel methods of screening for compositions which treat diabetes and/or diabetic complications via modulation of diabetes protein expression levels and activity.

In certain embodiments, the methods of this invention are used to identify compounds that can neutralize the effect of a diabetes protein. By “neutralize” is meant that activity of a protein and the consequent effect of over or underexpression on the cell is inhibited or blocked. The compound may do this by interfering with or enhancing the physiological function of the diabetes protein. Typically, the assay will comprise contacting a diabetes associated protein with a compound and determining the functional effect of the compound on the diabetes protein.

In other embodiments, having identified a particular gene with altered expression in diabetes, test compounds can be screened for the ability to modulate gene expression. This can be done on an individual gene level or by evaluating the effect of drug candidates on a “gene expression profile”. In one embodiment, the modulator suppresses a diabetes phenotype to achieve a normal tissue fingerprint. The preferred amount of modulation of the expression level will depend on the original change of the gene expression in normal versus tissue from the organism undergoing diabetes, with changes of at least 10%, 50%, 100-300%, and in some embodiments 300-1000% or greater. Thus, if a gene exhibits a 4-fold increase in diabetes tissue compared to normal tissue, a decrease of about four-fold is often desired; similarly, a 10-fold decrease in diabetes tissue compared to normal tissue often provides a target value of a 10-fold increase in expression to be induced by the test compound.

In some embodiments of the present invention, diabetes is induced in an animal, the animal or cells isolated from the animal are contacted with a candidate therapeutic agent, and the expression level of certain genes are monitored and compared to their expression level in control animals. Typically, the genes that are monitored are those that have been previously identified as altered in diabetic animal models and those where manipulation of expression levels is likely to correct defects in insulin secretion or insulin signalling or to treat diabetic complications. Expression levels are measured using the methods described in the Section VI

In one embodiment, the monitored gene is one that encodes a corticosteroid binding protein, a part of the corticosterone homeostasis system. Reduced levels of the corticosteroid binding protein generate an excess of corticosterone that induces gluconegenesis, thus exacerbating diabetes. Therefore, in order to identify drugs for treatment of diabetes, screens can be conducted to identify compounds that increase the amount of cortisone bound by the corticosteroid binding protein, either by identifying a compound that increases expression levels or one that enhances the protein's ability to bind corticosteroids.

Typically, a test compound is administered to an animal model with diabetes. By “administration” or “contacting” herein is meant that the candidate agent is administered in such a manner as to allow the agent to act upon the animal. In some embodiments, nucleic acid encoding a proteinaceous candidate agent (i.e., a peptide) may be put into a viral construct and administered as a gene therapy agent, such that expression of the peptide agent is accomplished, e.g., PCT US97/01019. In certain embodiments, the “test compound” is contacted with a cell isolated from an animal.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly treat diabetes or diabetic complication by modulating the activity or the expression of a diabetes sequence, e.g., a nucleic acid or protein sequence. Generally, a plurality of different agent concentrations are tested to obtain a differential response to the various concentrations. Typically, one of these concentrations serves as a negative control, i.e., at zero concentration or below the level of detection.

Measures of diabetes polypeptide activity or of diabetes protein expression levels can be performed using a variety of assays known to those of skill in the art. Therapeutic efficacy can be measured using standard clinical measures of diabetes severity. For example, a suitable physiological change, i.e. ability to maintain glucose homeostasis, can be used to assess the influence of a test compound on the polypeptides of this invention. Specific embodiments of the method are further described below.

B. Generating Animal Models of Diabetes

The diabetic animal models used in the methods of this invention can be generated using any method known to those of skill in the art.

Typically, diabetes is induced using streptozotocin, a nitrosylamino compound that selectively destroys the insulin-producing beta cells of the pancreas, induces peripheral insulin resistance, and alterations in insulin-dependent signal transduction.

C. Methods for Determining whether a Test Compound is a Therapeutic Compound for Diabetes

As described above, compounds for the treatment of diabetes can be identified by testing compounds for an ability to either modulate the activity of over/underexpressed diabetes proteins or for an ability to achieve normal expression levels of over/underexpressed diabetes proteins.

1. Methods for Detecting Diabetes Protein Activity

Based on knowledge of the function of the proteins over/underexpressed in response to SID, one of skill can use methods known to those of skill in the art to measure the activity of such proteins. Standard methods for detecting protein activity are described in Section VIII.

2. Methods for Monitoring Gene/Protein Expression Levels

Screening for the ability of compounds to modulate “gene expression profiles”, individual gene expression levels, and individual protein expression levels can be conducted via any method known to those of skill in the art, including those described in Section VI of this specification.

The amount of gene expression may be monitored using nucleic acid probes, or, alternatively, the gene product itself can be monitored, e.g., through the use of antibodies to the diabetes protein and standard immunoassays. Proteomics and separation techniques may also allow quantification of expression. In one embodiment, gene or protein expression monitoring of a number of entities, i.e., an expression profile, is monitored simultaneously. Such profiles will typically involve a plurality of those entities described herein.

In one embodiment, diabetes nucleic acid probes are attached to biochips as outlined above for the detection and quantification of diabetes sequences and expression monitoring is performed. Alternatively, PCR may be used. Thus, a series, e.g., of microtiter plate, may be used with dispensed primers in desired wells. A PCR reaction can then be performed and analyzed for each well.

D. High-Throughput Screening for Gene Transcription, Polypeptide Expression, & Polypeptide Activity

The assays to identify modulators are amenable to high throughput screening. Typical assays detect modulation of diabetes gene transcription, polypeptide expression, and polypeptide activity when test compounds are contacted with a cell isolated from an animal.

High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

E. Compounds to be Screened in Methods of this Invention

1. Combinatorial Libraries

In certain embodiments, combinatorial libraries of potential modulators will be screened for an ability to bind to a diabetes polypeptide, to modulate diabetes polypeptide activity, or to block expression of diabetes protein. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds.

In some embodiments, the drug screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

2. Proteins and Nucleic Acids as Potential Modulators

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. These can be libraries of bacterial, fungal, viral, and mammalian proteins, e.g., human protein. Particularly useful test compound will be directed to the class of proteins to which the target belongs, e.g., substrates for enzymes or ligands and receptors.

In one embodiment, modulators are peptides of from about 5 to about 30 amino acids, with from about 5 to about 20 amino acids, or from about 7 to about 15. The peptides may be digests of naturally occurring proteins as is outlined above, random peptides, or “biased” random peptides. By “randomized” or grammatical equivalents herein is meant that the nucleic acid or peptide consists of essentially random sequences of nucleotides and amino acids, respectively. Since these random peptides (or nucleic acids, discussed below) are often chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In another embodiment, the library is biased. That is, some positions within the sequence are either held constant, or are selected from a limited number of possibilities. In one embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, scrines, threonines, tyrosines or histidines for phosphorylation sites, etc.

Modulators of diabetes can also be nucleic acids, as defined above.

As described above generally for proteins, nucleic acid modulating agents may be naturally occurring nucleic acids, random nucleic acids, or “biased” random nucleic acids. Digests of procaryotic or eucaryotic genomes may be used as is outlined above for proteins.

The compounds tested as modulators can be any small chemical compound, or a biological entity, such as a protein, sugar, nucleic acid or lipid. Alternatively, modulators can be genetically altered versions of the genes. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

X. Pharmaceutical Administration & Compositions

In certain embodiments, the invention provides pharmaceutical compositions comprising the modulators identified through the assays described in the preceding section, combined with a physiologically acceptable excipient. As used herein, the term “modulator” refers to

A. Dosage

In one embodiment, a therapeutically effective dose of a modulator of a diabetes protein is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals. Thus the methods are applicable to both human therapy and veterinary applications. In a typical embodiment the patient is a mammal, usually a primate, and most typically, the patient is human.

B. Administration & Pharmaceutical Compositions

The administration of the modulators of diabetes protein of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the modulators may be directly applied as a solution or spray.

The pharmaceutical compositions of the present invention comprise a diabetes protein modulator in a form suitable for administration to a patient. In some embodiments, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that diabetes protein modulators (e.g., antibodies, antisense constructs, ribozymes, small organic molecules, etc.) when administered orally, should be protected from digestion. It is also recognized that, after delivery to other sites in the body (e.g., circulatory system, lymphatic system, or the tumor site) the diabetes modulators of the invention may need to be protected from excretion, hydrolysis, proteolytic digestion or modification, or detoxification by the liver. In all these cases, protection is typically accomplished either by complexing the molecule(s) with a composition to render it resistant to acidic and enzymatic hydrolysis, or by packaging the molecule(s) in an appropriately resistant carrier, such as a liposome or a protection barrier or by modifying the molecular size, weight, and/or charge of the modulator. Means of protecting agents from digestion degradation, and excretion are well known in the art.

The compositions for administration will commonly comprise a diabetes protein modulator dissolved in a physiologically acceptable carrier, typically an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gilman, The Pharmacological Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gilman, The Pharmacological Basis of Therapeutics, supra.

The compositions containing modulators of diabetes proteins can be administered for therapeutic or prophylactic treatments. In therapeutic applications, compositions are administered to a patient suffering from a diabetes or a diabetic complication (e.g., liver function abnormalities due to diabetes) in an amount sufficient to cure or at least partially arrest the diabetes or its complications. An amount adequate to accomplish this is defined as a “therapeutically effective dose.” Amounts effective for this use will depend upon the severity of the diabetes and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of diabetes or its complications in a mammal is referred to as a “prophylactically effective dose.” The particular dose required for a prophylactic treatment will depend upon the medical condition and history of the mammal, the particular type of diabetes or particular type of complication being prevented, as well as other factors such as age, weight, gender, administration route, efficiency, etc. Such prophylactic treatments may be used, e.g., in a mammal who has previously had diabetes to prevent a recurrence of the diabetes, or in a mammal who is suspected of having a significant likelihood of developing diabetes.

It will be appreciated that the present diabetes protein-modulating compounds can be administered alone or in combination with additional diabetes modulating compounds or with other therapeutic agent, e.g., other anti-diabetes, anti-diabetic complication agents or treatments.

XI. Assays to Determine Whether a Test Compound Induces Diabetes

In some embodiments, the diabetes-associated genes and proteins can be used as tools to establish that a compound for treating another disease does not induce diabetes or diabetic complications as a side effect. For example, a study may identify a drug that mimics the effects of caloric restriction. Methods for identifying such drugs are described in U.S. Pat. No. 6,406,853, issued to Steve Spindler. To determine whether such a drug also induces diabetes, the test compound can be administered to a mammal and the expression level of the diabetes genes and proteins can be measured to determine whether it is modulated in the same manner as it is in diabetes.

XII. Use of Diabetes-Associated Sequences and Proteins to Induce Animal Models of Diabetes

In certain other embodiments, the diabetes-associated sequences of this invention express proteins that play a critical role in the development of diabetes. The expression of such sequences can either be upregulated or downregulated to generate animal models of diabetes. Animal models of diabetes find use in screening for modulators of a diabetes-associated sequence or modulators of diabetes.

In some cases, the diabetes protein is underexpressed in diabetes. Transgenic animal technology including gene knockout technology, e.g., as a result of homologous recombination with an appropriate gene targeting vector, can be used to eliminate or decrease expression of the diabetes protein to induce diabetes. When desired, tissue-specific expression or knockout of the diabetes protein may be necessary.

It is also possible that the diabetes protein is overexpressed in diabetes. As such, transgenic animals can be generated that overexpress the diabetes protein. Depending on the desired expression level, promoters of various strengths can be employed to express the transgene. Also, the number of copies of the integrated transgene can be determined and compared for a determination of the expression level of the transgene. Animals generated by such methods find use as animal models of diabetes and are additionally useful in screening for modulators to treat diabetes.

XIII. Kits for use in Diagnostic and Prognostic Applications

For use in diagnostic, research, and therapeutic applications suggested above, kits are also provided by the invention. In the diagnostic and research applications such kits may include any or all of the following: assay reagents, buffers, diabetes-specific nucleic acids or antibodies, hybridization probes and/or primers, antisense polynucleotides, ribozymes, dominant negative diabetes polypeptides or polynucleotides, small molecule inhibitors of diabetes-associated sequences, etc. A therapeutic product may include sterile saline or another pharmaceutically acceptable emulsion and suspension base. Kits for screening for modulators of the expression levels of diabetes-associated proteins are also provided.

The kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

A wide variety of kits and components can be prepared according to the present invention, depending upon the intended user of the kit and the particular needs of the user. For example, diagnosis would typically involve evaluation of a plurality of genes or products. The genes will be selected based on correlations with important parameters in disease which may be identified in historical or outcome data.

### EXAMPLES

The following examples are offered to illustrate, but not to limit, the claimed invention.

### Example 1

This example illustrates the methods used to perform hepatic gene expression profiling of streptozotocin-induced diabetes.

Introduction

In the present study, microarrays containing probe sets for approximately 11,000 murine genes and expressed sequence tags (ESTs) were used to generate a profile of the liver response to SID. The liver was chosen for study because it is a major target of insulin action, and plays a pivotal role in blood glucose homeostasis. Our results showed that SID causes major alterations in the expression of genes involved in cytoprotective stress-responses, oxidative and reductive xenobiotic metabolism, growth and signal transduction, and carbohydrate, fat and protein metabolism.

Materials and Methods

Mice and treatment. Female, 8-month old Swiss-Webster mice were purchased from Jackson Laboratories. Diabetes was induced by three weekly intraperitoneal injections of streptozotocin (STZ; 10 mg/100 g body weight) in 50 mM sodium citrate, pH 4.5. Blood glucose measurements indicated that the mice were diabetic one week after the third injection. Only mice with blood glucose levels higher than 3 mg/ml were used. Mice injected with equivalent volumes of sodium citrate at the same times served as controls. Mice were sacrificed one week after the last injection. Mice were fasted for 24 h before sacrifice.

Measurement of Specific mRNA Levels. Total liver RNA was isolated from frozen tissue fragments using TRI Reagent (Molecular Research Center, Inc., Cincinnati, Ohio) as described previously (Cao et al., Proc. Natl. Acad. Sci. U.S. A. 98:10630-10635 (2001)). Specific mRNA levels were measured using the Affymetrix Mu11KsubA and Mu11KsubB high-density oligonucleotide arrays using standard Affymetrix protocols as described (Cao et al., Proc. Natl. Acad. Sci. U.S. A. 98:10630-10635 (2001)). Briefly, cDNA was prepared from total RNA using Superscript Choice System with a primer containing oligo(dT) and the T7 RNA polymerase promoter sequence. Biotinylated cRNA was synthesized from purified cDNA using the Enzo BioArray High Yield RNA Transcript Labeling Kit (Enzo Biochem), and CRNA purified using RNeasy mini columns (Qiagen, Chatsworth, Calif.). An equal amount of cRNA from each animal was separately hybridized to Mu11KsubA and Mu11ksubB high-density oligonucleotide arrays. The arrays were hybridized for 16 h at 45° C. After hybridization, arrays were washed, stained with streptavidin-phycoerythrin, and scanned using a Hewlett-Packard GeneArray Scanner.

Data Analysis and Statistics. Image analysis and data quantification were performed using the Affymetrix Microarray Suite 5.0. Affymetrix Mu11KsubA and Mu11KsubB arrays contain targets for more than 11,000 mouse genes and expressed sequence tags (ESTs). Each gene or EST is represented on the array by 20 perfectly matched (PM) oligonucleotides and 20 mismatched (MM) control probes that contain a single central-base mismatch. The signal intensities of PM and MM were used to calculate a discrimination score, R, which is equal to (PM−MM)/(PM+MM). A detection algorithm utilized R to generate a detection p-value and assign a Present, Marginal or Absent call using the Wilcoxon signed rank test (Wilcoxon F., Biometrics 1:80-83 (1945); Affymetrix, Inc., New Statistical Algorithms for Monitoring Gene Expression on GeneChip Probe Arrays, Technical Notes 1, Part No. 701097 Rev. 1 (2001)). Only genes that were “Present” in at least 2 out of 3 arrays per experimental group were considered for further analysis. A signal algorithm calculated the relative level of expression of each transcript using the One-Step Tukey's Biweight Estimate (Affymetrix, Inc., New Statistical Algorithms for Monitoring Gene Expression on GeneChip Probe Arrays, Technical Notes 1, Part No. 701097 Rev. 1 (2001)). This procedure generates a robust weighted mean that is relatively insensitive to outliers, even when they are extreme. Genes with a signal intensity lower than the mean array signal intensity in 2 or more of the 3 arrays in either experimental group were eliminated from the analysis. All arrays were scaled to a target intensity of 2500. To identify differentially expressed genes, each of the 3 control samples was compared with each of the 3 STZ-treated samples, resulting in 9 pairwise comparisons. Statistical analysis of these data was based on Wilcoxon's signed rank test (Wilcoxon F., Biometrics 1:80-83 (1945)). Difference values (PM-MM) between the control and STZ-treated arrays were used to generate a one-sided p-value for each set of probes. Default boundaries between significant and not significant p-values were used (Affymetrix, Inc., New Statistical Algorithms for Monitoring Gene Expression on GeneChip Probe Arrays, Technical Notes 1, Part No. 701097 Rev. 1 (2001)). We considered genes to have changed expression in the STZ-treated group if the number of increase or decrease calls was six or more of the nine pairwise comparisons, and an average fold change, derived from all nine possible pairwise comparisons, was 1.8-fold or greater. Empirically we found that these criterion identified gene expression changes which were reliably verified by Northern blotting (see below). Thresholds between 1.6 and 2.0 are common (Cao et al., Proc. Natl. Acad. Sci. U.S. A. 98:10630-10635 (2001); Kaminski et al., Proc. Natl. Acad. Sci. U.S.A. 97:1778-1783(2000)). Gene names were obtained from the Jackson Laboratory Mouse Genome Informatics database (May 1, 2002).

Validation by Northern Blotting. The expression of a total of ten genes was examined by Northern blotting using total hepatic RNA purified from the mice used in the microarray studies and, in addition, three more mice per group that were identically treated as a part of the same study, for a total n=6 for each group. Twenty μg of total RNA was separated on 1% agarose gels containing 17% formaldehyde. RNA was transferred to GeneScreen Plus (NEN Life Science Products, Boston, Mass.). Probe labeling was performed as described (Dhahbi et al., J. Gerontol. A Biol. Sci. Med. Sci. 53:B180-B185 (1998)). A mouse corticosteroid binding globulin probe was generated by PCR from mouse liver cDNA using the primers 5′-ccagtacctcaattcccttctcc-3′ and 5′-cccacatctgccagcacatc-3′. A 1-kb cDNA of murine heat shock 70 kD protein 8 cDNA was excised with Pst I from phsc1.5 and used as a probe (Giebel et al., Dev. Biol. 125:200-207 (1988)). A Syndecan 4 probe was amplified by PCR from mouse liver cDNA using the primers 5′-tccctgaagtgattgagccc-3′ and 5′-gggagagagagagagagagaga-3′. A 3′-noncoding fragment of cytochrome P450, 1a2, aromatic compound inducible, was excised by Bam HI/Hind III digestion of pP₃450FL (Kimura et al., Mol. Cell. Biol. 6:1471-1477 (1986)). A mouse solute carrier family 22 (organic cation transporter), member I probe was amplified by PCR using the primers 5′-ctcttgtgctgtgctgtacc-3′ and 5′-cctccttcctctctccactc-3′. A mouse histidine ammonia lyase probe was generated by PCR using the primers 5′-aagccctcagggtcgtcgagcacgt-3′ and 5′-ctgggttcagccactgcattgctgc-3′. The mannose-6-phosphate receptor, cation dependent gene probe was generated by PCR from a mouse cDNA library using the primers 5′-gcaatgacaaggagacagtgg-3′ and 5′-agggcaaggtgagagatggg-3′. A mouse ornithine decarboxylase antizyme probe was generated by PCR using the primers 5′-aggacagttttgcagctctcctaga-3′ and 5′-ctgggttcagccactgcattgctgc-3′. The entire coding fragment of CCAAT/enhancer binding protein beta (C/EBPβ) cDNA was used as a probe [Cao et al., Genes Dev. 5:1538-1552 (1991)]. A 1.5 kb probe for the hamster heat shock 70 kD protein 5 (glucose-regulated protein, 78 kD) gene was obtained by EcoRl/PstI digestion of p3C5 (A. R. Day and A. S. Lee, DNA 8:301-310 (1989)). Specific mRNA levels were normalized as described (Cao et al., Proc. Natl. Acad. Sci. U.S. A. 98:10630-10635 (2001)). Blots were analyzed and mRNA levels quantified using Molecular Dynamics PhosphorImager and ImageQuaNT software (Molecular Dynamics, Sunnyvale, Calif.).

Results and Discussion

The gene expression profile of liver tissue from control and STZ-treated mice was compared in order to assess the mRNA levels of more than 11,000 known genes and ESTs. SID was found to be associated with a change in expression of 47 known genes (Table 1).

The Affymetrix oligonucleotide microarrays yield quantitative data with a high degree of reliability as judged by similarities in Northern blot and Affymetrix analysis of mRNA samples from an overlapping set of mice (Cao et al., Proc. Natl. Acad. Sci. U.S. A. 98:10630-10635 (2001)). The effectiveness of the analytical criteria used for the array data was judged by comparison to Northern blots of 6 SID and 6 normal mice. Five genes were chosen from among those which changed expression with STZ treatment (Table 1). The changes in the expression of four of these genes were similar as to direction and magnitude using the two techniques (FIG. 1). Five genes that did not change expression with STZ on the microarrays also did not change expression when analyzed using Northern blotting (data not shown). Together these data show that the analysis method used is capable of identifying differentially expressed genes.

In conclusion, high-density DNA array technology has been used to identify gene expression patterns that underlie the biochemical, morphological and functional alterations in the liver physiology produced by SID. These findings identify changes in gene expression that contribute to the pathophysiology of diabetes. These changes include alterations in the expression of genes associated with carbohydrate, lipid and protein homeostasis. They also include disregulation of genes associated with cell growth and signal transduction, and the alteration of antioxidant-related gene expression. The expression of genes coding for differentiated functions of the liver, including the detoxification pathways, also were altered. The gene profiles reported in this study can provide targets for gene therapy and rational drug development.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of detecting diabetes in a patient, said method comprising detection of the expression level of at least one polynucleotide selected from the group consisting of: polynucleotides listed in Table 1.

2. The method of claim 1, wherein said method further comprises detection of the expression level of a second polynucleotide selected from the group consisting of: polynucleotides listed in Table 1.

3. The method of claim 1, wherein said method comprises detection of at least one polynucleotide selected from the group consisting of: a histidine lysase gene, a beta CCAAT/enhancer binding protein (CC/EBP), solute carrier family 22 (member 1), Cyt P450 1a2 (aromatic compound), and a corticosteroid binding protein.

4. The method of claim 1, wherein said expression level is detected by an oligonucleotide array.

5. The method of claim 1, wherein said expression level is detected by a Northern blot.

6. The method of claim 1, wherein said diabetes is Type I diabetes.

7. The method of claim 1, wherein said diabetes is Type II diabetes.

8. A method of treating diabetes or diabetic complications, wherein said method comprises modulating the expression level of at least one polynucleotide selected from the group consisting of: polynucleotides listed in Table 1.

9. The method of claim 8, wherein said diabetes is Type II diabetes.

10. The method of claim 8, wherein said diabetic complications are selected from the group consisting of: alterations in xenobiotic metabolism, retinopathy, nephropathy, neuropathy, and hepatic functional abnormalities.

11. The method of claim 10, wherein said diabetic complications are hepatic functional abnormalities.

12. The method of claim 13, wherein said hepatic functional abnormalities are selected from the group consisting of: loss of hepatic insulin sensitivity; excessive protein breakdown; reduction of protein synthesis; excessive hepatic glucose production (gluconeogenesis); reduction in cell growth and regeneration; excessive cell death (apoptosis); serum dyslipidemia resulting from altered hepatic lipid and protein metabolism; altered drug, metabolite, and xenobiotic metabolism; enhanced oxidative damage and cellular stress; and enhanced inflammatory and disregulated immune responsiveness.

14. The method of claim 8, wherein said modulation comprises reducing the expression level of said polynucleotide.

15. The method of claim 8, wherein said modulation comprises increasing the expression level of said polynucleotide.

16. The method of claim 8, wherein said modulation comprises the use of anti-sense molecules, ribozymes, and small interfering RNAs.

17. A method of treating diabetes or diabetic complications, wherein said method comprises modulating the activity of at least one polypeptide encoded by a polynucleotide selected from the group consisting of: polynucleotides listed in Table 1.

18. An assay for identifying a compound that modulates a polypeptide encoded by a polynucleotide selected from the group consisting of: polynucleotides listed in Table 1, said assay comprising the steps of:
(a) contacting a compound with said polypeptide; and
(b) determining the functional effect of the compound upon said polypeptide.

19. A high-throughput drug screening assay, wherein said method comprises the steps of:
(a) contacting a cell isolated from a mammal with streptozotocin-induced diabetes with a test compound;
(b) measuring the expression level of a polynucleotide selected from the group consisting of polynucleotides listed in Table 1;
(c) comparing the expression level of said polynucleotide in a cell contacted with a test compound to the expression level of said polynucleotide in a cell from a control mammal,
wherein said test compound that modulates the expression level of said polynucleotide is a candidate for the treatment of diabetes or diabetic complications.

20. A high-throughput assay for determining whether a test compound induces diabetes, wherein said method comprises the steps of:
(a) contacting a cell isolated from a mammal with a test compound;
(b) measuring the expression level of a polynucleotide selected from the group consisting of: polynucleotides listed in Table 1; and
(c) determining whether said test compound modulates the expression level of said polynucleotide in the same manner as diabetes, wherein said test compound that modulates the expression level of said polynucleotide in the same manner as diabetes is a compound that induces diabetes.

21. A drug screening assay, wherein said method comprises the steps of:
(a) administering a test compound to a mammal with streptozotocin-induced diabetes;
(b) measuring the expression level of a polynucleotide selected from the group consisting of: polynucleotides listed in Table 1; and
(c) comparing the expression level of said polynucleotide in a mammal contacted with a test compound to the expression level of said polynucleotide in a control mammal;
wherein said test compound that modulates the expression level of said polynucleotide is a candidate for the treatment of diabetes or diabetic complications.

22. A assay for determining whether a test compound induces diabetes, wherein said method comprises the steps of:
(a) administering a test compound to a mammal;
(b) measuring the expression level of a polynucleotide selected from the group consisting of: polynucleotides listed in Table 1; and
(c) determining whether said test compound modulates the expression level of said polynucleotide in the same manner as diabetes,
wherein said test compound that modulates the expression level of said polynucleotide in the same manner as diabetes is a compound that induces diabetes.

23. A pharmaceutical composition comprising a compound identified by the assay of claim 17, 18, or 21 and a physiologically acceptable excipient.
